(19) United States
(12) Patent Application Publication
(10) Pub. No.: US 2021/0255163 A1
(43) Pub. Date: Aug. 19, 2021

(54) METHOD FOR ASSESSING THE COMPATIBILITY OF PRODUCTION FLUID ADDITIVES

(71) Applicant: Anpera Technologies Limited, Edinburgh (GB)

(72) Inventors: Scott RANKIN, Whitburn, Bathgate (GB); Andrew OSNOWSKI, Stepps, North Lanarkshire (GB); Emma PERFECT, Edinburgh (GB); Harry GROVER, Edinburgh (GB); Fiona Carson, Dunbar (GB)

(21) Appl. No.: 17/252,417

(22) PCT Filed: Jun. 17, 2019
(86) PCT No.: PCT/GB2019/051685

§ 371 Date: Dec. 15, 2020

(30) Foreign Application Priority Data
Jun. 18, 2018 (GB) 1809982.0

(51) Int. Cl. G01N 33/28; G01N 31/22; C09K 8/54; G01N 1/38

(52) U.S. Cl. CPC G01N 33/2882; G01N 31/22; G01N 15/14; G01N 1/38; C09K 8/54

(57) ABSTRACT

A method of determining the suitably of corrosion inhibitors, or other additives in the presence of corrosion inhibitor, for a given fluid environment. The method including determining if there is a difference in the presence or level of micelles between a fluid sample to which corrosion inhibitor has been added either alone or to which corrosion inhibitor and at least one additional fluid additive or additives have been added. The method can be used to determine the compatibility of fluid additives.

The present invention relates to methods of determining the suitability of a corrosion inhibitor for different fluid environments and/or determining which additional fluid additives are suitable for use with a particular corrosion inhibitor. More particularly, the fluid environment is an oil and gas production fluid environment.

Corrosion inhibitors are often the first line of defense against internal corrosion in the oil and gas industry. The most common class of corrosion inhibitor used in the oilfield are amphiphilic surfactant molecules. They are also commonly used in oil and gas processing and petrochemical industries. When introduced into a pipe, these compounds partition to interfaces where the opposite electrostatic properties of each part of the molecule create energetic repulsions. In practice, the molecules adsorb to the surface of the pipe, and form ordered structures on the surface thereby creating a protective film. Quaternary amines, phosphate esters, amides, imidazolines, pyridinium and carboxylates are all commonly found in commercial corrosion inhibitor formulations, along with synergists and other components tailored to suit the fluidic properties of the specific production system.

Identifying the best inhibitor and dosage for any given field, typically known as chemical ‘qualification’, is a significant task. Examples of best practice can be found from NACE (National Association of Corrosion Engineers) and ASTM (American Society for Testing and Materials) and individual oil and gas operators may have their own procedures (ASTM 0170 G184 G202 G185 G208; NACE Publication 1D196; Webster et al., 1996; Papavinasam, 2014; Sonke and Grimes, 2016 and 2017).

All oil and gas assets vary in the chemistry of the fluids as well as how important it is to manage corrosion risk, i.e. their criticality. Testing therefore needs to be tailored and there is no one test that can select the most appropriate inhibitor. Broadly speaking, inhibitors are evaluated for: efficiency, suitability under operating conditions, compatibility and emulsions. For assets that are highly dependent on chemical protection, relevant laboratory tests may include: static linear polarisation resistance (LPR), rotating cylinder electrode (RCE), stirred autoclave, rotating cage autoclave, jet impingement, flow loop, and field trials. These may also be supported with interfacial tension testing, partitioning assessment, chemical compatibility and persistency.

Chemical analyses are often undertaken to ascertain changes in the level of the corrosion inhibitor during and after such tests. Current residual monitoring methods include simple methods, such as the methyl orange/chloroform colorimetric-complex test, or more complex methods, such as those using liquid chromatography-mass spectrometry. The former tends to suffer from interferences, including from oil or high salt concentration and not all chemicals are suitable. The latter require specialist equipment, highly skilled scientists and are often not possible to use by the wider community due to the chemicals being proprietary commercial formulations with such analyses being prohibited by the sellers. This point is even more relevant when different production chemicals for one field are sourced from different companies, and where proprietary information cannot be shared. It can be very challenging to carry out compatibility tests in such circumstances.

One important aspect of how a corrosion inhibitor will work in a production environment is how it functions in the presence of other production fluid additives. Various additives are added to production fluids. For example, corrosion inhibitors, biocides, foamers, defoamers, paraffin control agents, emulsifiers, demulsifiers, anti-swelling agents, hydrate inhibitors, anti-caking agents, scale dissolvers, wetting agents, and wax control agents. These additives are added at various levels depending on their function and expense.

Drilling and produced fluids can therefore end up as a cocktail of chemicals, water, salt and hydrocarbons. The chemicals in the cocktail can interact, which can impair function of the chemistries and risk issues with the infrastructure, causing shut downs and deferred production.

There are examples in the literature of incompatibility and recommendations have been made that incompatibility testing should be undertaken. For example, in Skogsberg and Miglin, 2001, the authors noted it was challenging to find a combination of corrosion and scale inhibitor that gave satisfactory corrosion and scale control and the importance of doing compatibility testing, e.g. corrosion testing where the scale inhibitor is present and vice versa. In Menendez et al., 2014, corrosion inhibitors were noted in interference with hydrate inhibition.

Nevertheless, current testing of chemicals can be laborious and time consuming. A simple corrosion inhibitor test, such as a mass loss corrosion rate test or static LPR (linear polarisation resistance) test typically take many hours or days, and tests that better reflect actual field conditions, such as flow loop tests, typically take many days. Scale inhibitor and hydrate inhibitor tests also take hours or days.

In light of this, compatibility testing may not be widely conducted. Or rather, significant effort is put into testing a particular corrosion, scale or hydrate inhibitor for a field, but not of chemicals in the presence of other production chemicals. The assumption is frequently made that if a chemical works in isolation, it will work well when mixed in a fluid with other chemicals.

Partitioning is another important consideration when determining which corrosion inhibitor to use for a given production fluid. Partitioning refers to the behaviour the corrosion inhibitor shows as it moves between the hydrocarbon and aqueous phases of field fluids. It is a very complex phenomenon, reflecting the multicomponent nature of the corrosion inhibitor formulations and the variable conditions the chemical may face in the field. It is relevant as it influences ‘availability’ of the chemical to protect the system. To give an example, for a corrosion inhibitor to protect against aqueous corrosion from export fluid, some of the protective chemical needs to be retained in the aqueous phase, and cannot all exist in the hydrocarbon phase.

Water:Octanol partitioning of chemicals for the oil and gas industry is tested for regulatory compliance purposes in the UK and Norway. The analytical methods which may be used are photometry, gas chromatography and high performance liquid chromatography. It is not considered a representative test for partitioning in field fluids.

The methyl orange/chloroform method, which detects the amount in the water phase after injecting the chemical to the oil-water mixture, agitating, separating and sampling, can be used. As noted above, this method suffers from interferences (not all chemicals are suitable, oil and salt can interfere).

Solid particles in a production fluid may also affect the function of a corrosion inhibitor. In an ideal world, only the internal diameter of the pipeline would present a binding surface for corrosion inhibitor; in reality this is not the case. Solids originating from formation residues, corrosion by-products and scale formation can all provide secondary binding sites for corrosion inhibitor, leading to ‘parasitisation’ of the chemical and decreased system protection through insufficient inhibitor concentrations for full protection of pipeline walls. Work by Smith et al. indicates that solids in dirty systems can act as a sink for available corrosion inhibitor. This may lead to corrosion control issues where optimal dosing does not consider secondary binding sites.

Limited work is conducted in chemical qualification work to investigate the effects solids may have on corrosion inhibitor parasitisation.

A simpler test for determining the compatibility of corrosion inhibitors for a given production fluid environment would allow better assessment of those field conditions which can influence corrosion inhibitors. These include: the partitioning of chemical species to the hydrocarbon, or aqueous phase; their interaction with other production chemicals; the parasitisation of the chemical to solids, which may exist in the system.

Applicant's own publication WO2010/007397 describes an improved methodology for the detection of production fluid additives, including corrosion inhibitor, that uses the relationship between an additive at its effective dose and the presence of micelles. Micelle detection methods do not require detailed information on the chemical composition of the corrosion inhibitor formulations and the need for an extraction process is minimised. Therefore, these methods can be performed quickly and simply.

What is needed is a simple method for determining the suitability of a corrosion inhibitor for different production, transportation and storage environments or the suitability of other chemical additives with particular corrosion inhibitors. A simpler method would stimulate more testing and allow more screening to take place, which better reflects the systems themselves, with the end result being the identification of chemicals, and dosage levels, better suited to field situations. Ultimately, more suitable chemicals will be identified and production and processing systems better protected as a result.

It is an objective of the present invention to seek to mitigate problems such as those described above.

For the purposes of this patent application, the term “additive” may include intentional additives such corrosion inhibitors, biocides, foamers, defoamers, paraffin control agents, emulsifiers, demulsifiers, anti-swelling agents, hydrate inhibitors, anti-caking agents, scale dissolvers or inhibitors, wetting agents, or wax control agents as well as unintentional additives such as solids that are in the fluid. Examples of such solids include sand, kaolin, limestone, illite, iron (II)(III) oxide, iron(II) sulfide, barium sulfate, or calcium sulfate.

For the purposes of this patent application, the term “concentration series” refers to an increasing concentration of a chemical, for example a corrosion inhibitor, being present in a fluid sample. A concentration series may be formed by adding increasing concentration of a chemical to each of separate identical samples and then monitoring each of the samples to record a marker signal. Alternatively, a concentration series may be formed by sequentially increasing the concentration of a chemical in the same fluid sample and monitoring the sample to record a marker signal after each increase in compound concentration.

For the purposes of this patent application, the term “water cut” refers to the ratio of aqueous phase to hydrocarbon phase in a fluid.

For the purposes of this patent application, the term “production fluid” refers to the fluid mixture of oil, gas, solids, and aqueous phase present in a conducting and containment system used to screen test, produce, transport and process oil and/or gas and their products.

For the purposes of this patent application, the term “reagent(s)” refers to chemicals or enzymes that react, interact, or contribute to the detection of a micelle, or micelles, in order to produce a detectable signal.

The term “extraction” refers to transferring the treatment chemical from a fluid that is immiscible with water to an aqueous phase. The term “separation” means the physical separation of the two phases into separate vials.

According to a first aspect of the invention, there is provided a method of determining the suitability of corrosion inhibitors for a given fluid environment comprising determining if there is a difference in the presence of micelles between a fluid sample to which corrosion inhibitor alone has been added and a fluid sample to which corrosion inhibitor and a second fluid additive has been added. A screening test based on micelle detection brings simplicity and greatly reduces the time required compared with current techniques. The present invention can be applied to micelles in the aqueous phase or, so called, reverse micelles in the hydrocarbon phase. The present invention also provides information on the corrosion inhibitor concentration which meets or exceeds the critical micelle concentration, rather than information on the concentration of a very specific component. There is a relationship, albeit a complex one, between micelle formation and adsorption (Chandra et al, 2018) and so micelles, as opposed to distinct chemical components are relevant for qualification. This provides a qualitative indication of how conditions introduced in qualification testing influences the corrosion inhibitor and in turn allows decisions to be made as to its suitability to the field.

The present invention relates to determining the suitability of corrosion inhibitors for a given fluid environment and/or determining the suitability of additional fluid additives for a given corrosion inhibitor. It may be the case that certain corrosion inhibitors are not suitable for certain fluid environments and it may alternatively be the case that certain corrosion inhibitor and additional additives are not suitable for use together in certain fluid environments. Therefore, the present invention allows a user to determine which corrosion inhibitor and additional fluid additive pairings will work together at their optimal performance for a given fluid additive.

In one embodiment, multiple tests on different aspects of a corrosion inhibitor may be carried out in order to determine the full compatibility of a given corrosion inhibitor in a given fluid environment. For example, one or more tests comprising partitioning of chemical species to the hydrocarbon, or aqueous phase; their interaction with other production chemicals; or the parasitisation of the chemical to solids. In another embodiment, a user may wish to investigate only one, or a selection of, aspect of a corrosion inhibitor. For example, one or more tests selected from a list consisting of the partitioning of chemical species to the hydrocarbon, or aqueous phase; their interaction with other production chemicals; or the parasitisation of the chemical to solids.

In an alternative embodiment, tests on additional fluid additives may be carried out in order to determine which additional fluid additives are suitable for use with a particular corrosion inhibitor, in a given fluid environment. This is advantageous in circumstances where the user has no flexibility in the choice of corrosion inhibitor used in the fluid environment.

In one embodiment, the presence of micelle in the fluid sample may be determined by adding marker solution comprising an optically detectable marker in which the marker solution is a single reagent that produces an optically detectable signal. Examples of the types of reactions used as the marker solution to generate an optical signal include fluorgenic, chromogenic, luminescent, infrared or Raman-active. The optical signal may be detected using fluorescence, chemiluminescence, colourimetry or UV-visible, IR or Raman spectroscopy or by eye.

In an alternative embodiment, the presence of micelle in the fluid sample may be determined by adding marker solution comprising an optically detectable marker, in which the marker solution is a combination of a first reagent and a second reagent. In this embodiment the first reagent interacts with the corrosion inhibitor, alone, or in the presence of a second fluid additive, where the interaction may be a reaction or a physical interaction, such as move from hydrophobic to hydrophilic environment, to produce a detectable signal. Examples of the types of detectable signal include fluorogenic, chromogenic, luminescent, infrared or Raman-active. An advantage of using signal detection of the types listed above is that detection can be instantaneous and/or automated, allowing for a high system throughput which in turn reduces running costs.

Preferably, an aqueous fluid is added to the fluid sample after the step of obtaining a fluid sample to which corrosion inhibitor is to be added, to form a predetermined ratio of aqueous fluid to hydrocarbon fluid. This feature is advantageous because it is a straightforward way of accurately recreating the system in the real fluid environment, which can include difference in the type of oil, salinity, pH, levels of aromatic components or aliphatics, or water chemistries.

Preferably, the fluid additive used in the method of the present invention is an unintended fluid additive and comprises solid particles. The use of this type of fluid additive in the method of the present invention is advantageous because it will encourage and make investigation into the effects solids may have on corrosion inhibitor parasitisation more accessible to a user.

Preferably, the fluid sample is taken from, or representative of, a fluid environment of a conducting and containment system used to screen test, produce, transport and process oil and/or gas and their products. The advantage of this feature is that it contributes to the provision of results which can be confidently applied in the field.

Detection of corrosion inhibitor may be performed using chromogenic agents that react with aromatic groups, unsaturated bonds, hydroxyls or amine groups are used. Preferably, NanoOrange®. Nile Red (9-diethylamino-5-benzo[α]phenoxazinone), Laurdan (6-Dodecanoyl-2-Dimethylaminonaphthalene), FM 4-64 (N-(3-Triethylammoniumpropyl)-4-(6-(4-(Diethylamino) Phenyl) Hexatrienyl) Pyridinium Dibromide) and 2,6-ANS (2-Anilinonaphthalene-6-sulfonic acid) are used. If other fluid additives are to be detected, then other reagents may be required. For example, meropolymethines, pyridinium-N-phenolate betaines, phenoxazones, N,N-dialkylaminonaphthalenes, N,N-dialkylaminostyrenes, N,N-dialkylaminonitrobenzenes, coumarins, N,N-dialkylindoaniline, vinylquinoliums, arylaminonaphthalene sulfonates.

The optical signal may be detected using fluorescence, chemiluminescence, colourimetry or UV-visible, JR or Raman spectroscopy or by eye. The signal may be detected using a fluorescence detector, luminescence detector, Raman detector, optical microscope, CCD camera, photographic film, fibre-optic device, photometric detector. MEMS device, single photon detector, spectrophotometer, chromatography system or by eye.

The invention will now be described by way of example with reference to the figures.

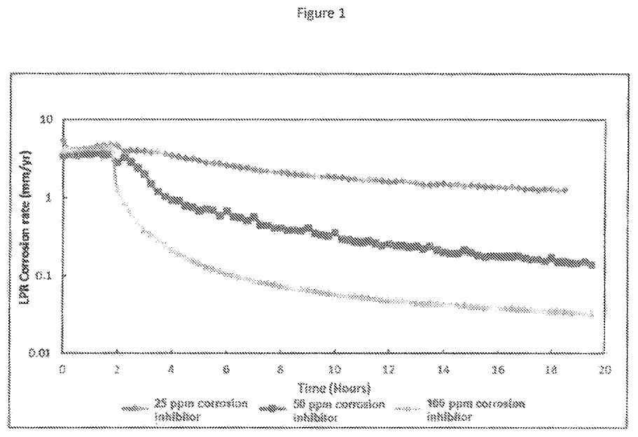

FIG. 1 is a graph of LPR corrosion rate for corrosion inhibitor 30-60% fatty acid amine condensate, acetates solution containing 10-30% 2-Butoxyethanol at 25 ppm, 50 ppm, and 100 ppm;

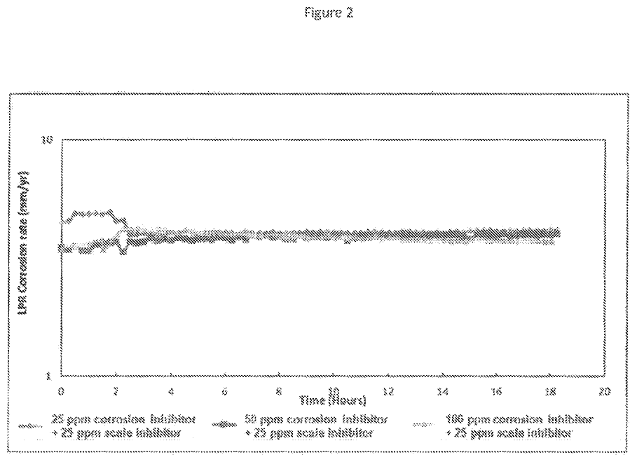

FIG. 2 is a graph of LPR corrosion rate for corrosion inhibitor 30-60% fatty acid amine condensate, acetates solution containing 10-30% 2-Butoxyethanol at 25 ppm, 50 ppm, and 100 ppm in the presence of a 25 ppm dose of scale inhibitor;

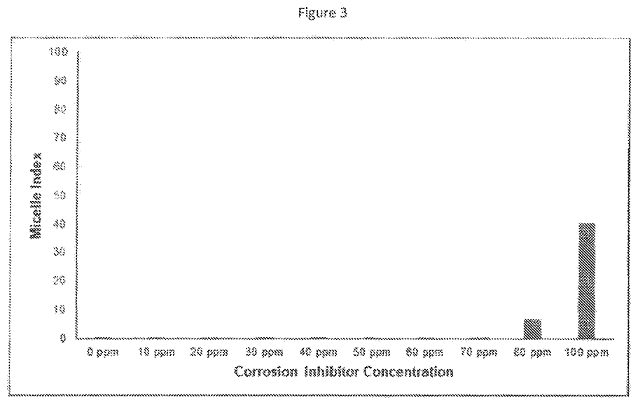

FIG. 3 is a graph of a concentration series of surfactant-containing corrosion inhibitor formulated in sodium thiosulphate, IPA (1-10%), glycol and sodium chloride in brine;

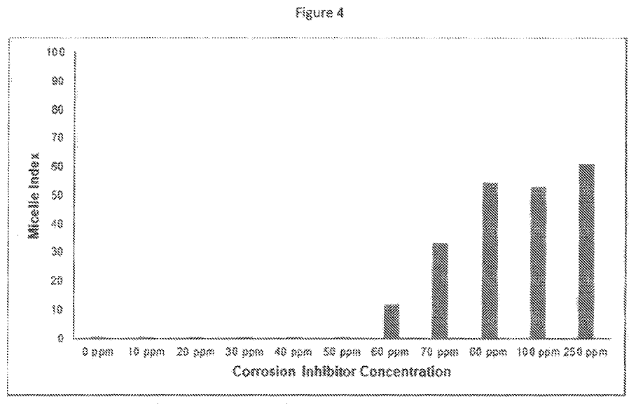

FIG. 4 is a graph of a concentration series of surfactant-containing corrosion inhibitor formulated in sodium thiosulphate, IPA (1-10%), glycol and sodium chloride in 9:1 brine:petroleum ether;

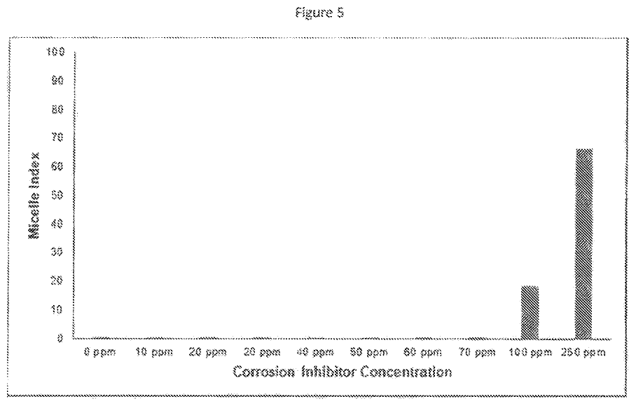

FIG. 5 is a graph of a concentration series of surfactant-containing corrosion inhibitor formulated in sodium thiosulphate. IPA (1-10%), glycol and sodium chloride in 9:1 brine:peregrino oil;

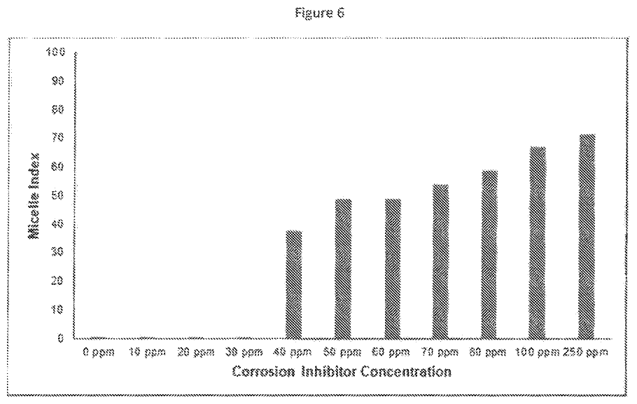

FIG. 6 is a graph of micelle index for surfactant-containing corrosion inhibitor formulated in sodium thiosulphate. IPA (1-10%), glycol and sodium chloride in 6:4 brine:petroleum ether;

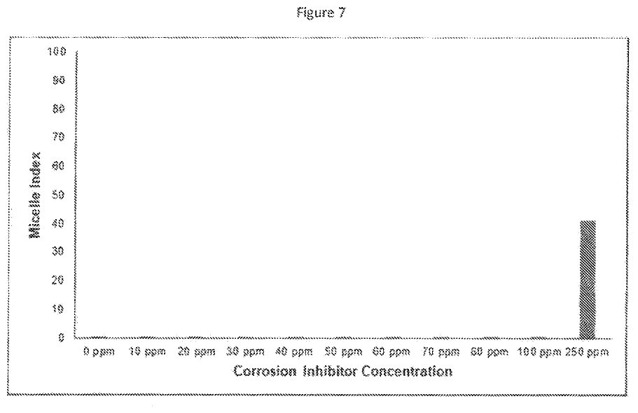

FIG. 7 is a graph of micelle concentration of surfactant-containing corrosion inhibitor formulated in sodium thiosulphate, IPA (1-10%), glycol and sodium chloride in 6:4 brine:oil;

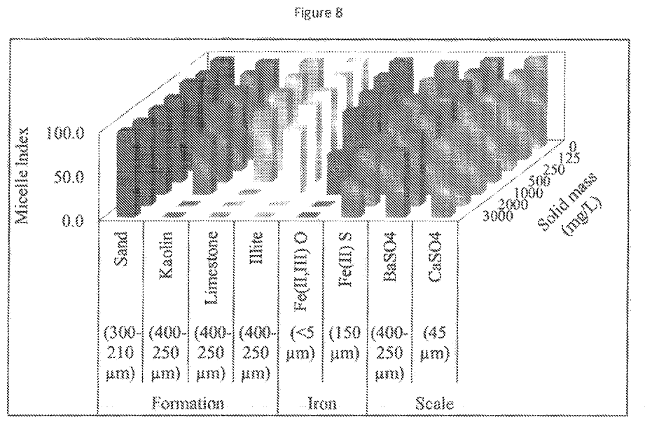

FIG. 8 is graph of corrosion inhibitor 1—1M NaCl (21° C. 20 hours equilibration):

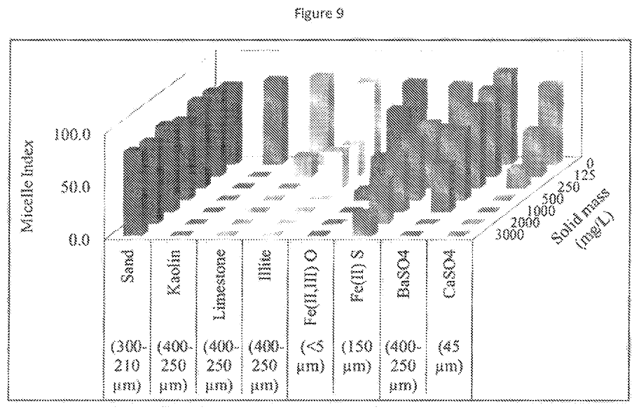

FIG. 9 is a graph of corrosion inhibitor 2—1M NaCl (21° C., 20 hours equilibration);

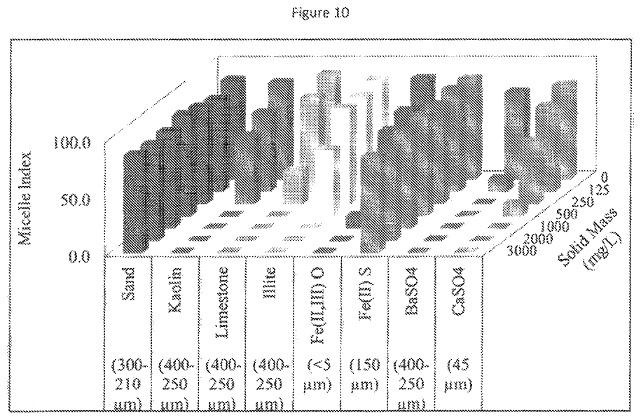

FIG. 10 is a graph of corrosion inhibitor 3—1M NaCl (21° C. 20 hours equilibration); and

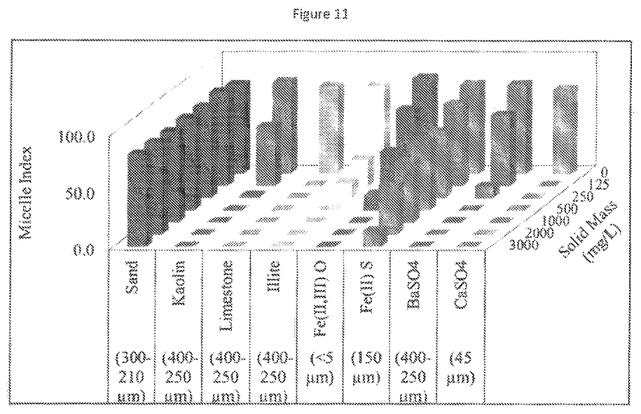

FIG. 11 is a graph of corrosion inhibitor 4—1M NaCl (21° C., 20 hours equilibration).

### EXPERIMENT 1: COMPATIBILITY OF CORROSION INHIBITORS AND A SECOND FLUID ADDITIVE

Samples were taken from LPR tests corrosion rate in which scale inhibitors and/or corrosion inhibitors had been dosed. Sample bottles were filled to the top and lids replaced as soon as possible in order to minimise oxygen ingress. The average micelle result from each set of tests is shown.

The conditions for the LPR tests were as follows:

| | |
|---|---|
| Temperature | 50° C. |
| Brine | 1 M NaCl (100% aqueous) |
| Gas | CO₂ |
| Pre-Corrosion | 2 hours |
| Sweep Rate: | −10 mV to +10 mV at |
| | 10 mV.min⁻¹ |
| Coupon material: | C1018 |
| Coupon Type | X65 Carbon Steel |

The test was run for a minimum 16 hours

Two hours after pre-corrosion, cells were dosed with 25, 50 or 100 pm of a 30-60% fatty acid amine condensate, acetates solution containing the corrosion inhibitor 10-30% 2-Butoxyethanol. Immediately after the corrosion inhibitor dose a 25 ppm dose of scale inhibitor was added. LPR measurements were continued until the following morning.

Corrosion inhibitor after 2 hours pre-corrosion and then immediately following the corrosion inhibitor dose, the scale inhibitor dose was added. To determine the presence of micelles, 1.98 mL of sample was taken, to which 20 μL of 0.1 mM Nile Red was added, and optical signal determined.

As shown in Table 1 and FIG. 1, no micelles were present in the samples at 25 and 50 ppm corrosion inhibitor and the final corrosion rate is higher than that of the 100 ppm sample. Micelles are present in the 100 ppm sample and a final corrosion rate of 0.03 mm/yr is achieved.

TABLE I
LPR corrosion rate for corrosion inhibitor 30-60%
fatty acid amine condensate, acetates solution containing
10-30% 2-Butoxyethanol at 25 ppm, 50 ppm, and 100 ppm.

| | | Peak corrosion | | | |
|---|---|---|---|---|---|
| | | rate before | Final | Inhibitor | |
| Test | Inhibitor | inhibitor dosage | Corrosion | Efficiency | |
| No. | combination | (mm/yr) | rate (mm/yr) | (%) | Micelle? |
| 1 | 25 ppm | 4.58 | 1.25 | 72.83 | No |
| 2 | 50 ppm | 3.22 | 0.14 | 95.69 | No |
| 3 | 100 ppm | 4.47 | 0.03 | 99.26 | Yes |

As shown in Table 1 and FIG. 1, the results shows that a concentration of 100 ppm of the corrosion inhibitor comprising 30-60% fatty acid amine condensate, acetates solution containing 10-30% 2-Butoxyethanol is the optimal concentration for protection, as it has reached the CMC and micelles were detected.

TABLE 2
LPR corrosion rate for corrosion inhibitor 30-60% fatty acid
amine condensate, acetates solution containing 10-30%
2-Butoxyethanol at 25 ppm, 50 ppm, and 100 ppm
in the presence of a 25 ppm dose of scale inhibitor.

| | | Peak corrosion | Final | | |
|---|---|---|---|---|---|
| | | rate before | Corrosion | Inhibitor | |
| Test | Inhibitor | inhibitor dosage | rate | Efficiency | |
| No. | combination | (mm/yr) | (mm/yr) | (%) | Micelle? |
| 4 | corrosion | 4.54 | 4.17 | 8.08 | No |
| | inhibitor | | | | |
| | 25 ppm & | | | | |
| | scale inhibitor | | | | |
| | 25 ppm | | | | |
| 5 | corrosion | 3.72 | 4.03 | −8.28 | No |
| | inhibitor | | | | |
| | 50 ppm & | | | | |
| | scale inhibitor | | | | |
| | 25 ppm | | | | |
| 6 | corrosion | 4.00 | 3.74 | 6.39 | No |
| | inhibitor 100 | | | | |
| | ppm & scale | | | | |
| | inhibitor | | | | |
| | 25 ppm | | | | |

As shown in Table 2 and FIG. 2, the results show that the scale inhibitor at 25 ppm rendered the corrosion inhibitor completely inefficient for corrosion protection at any of the dose rates tested. This inefficiency was reflected in micelle presence, with no micelles detected in any of the samples when scale inhibitor was present.

### EXPERIMENT 2: DETECTION OF CORROSION INHIBITOR PARTITIONING BETWEEN AQUEOUS AND HYDROCARBON PHASES

The partitioning of an amine derivative surfactant in 10-20% ethanol in a heavy field oil were tested. The oil was stored at 60° C. for at least four hours prior to analysis and was mixed regularly to ensure homogeneity. The aqueous phase was based on a synthetic field brine and consisted of the following quantities of each salt dissolved in 1 L of deionised water.

TABLE 3
Composition of synthetic field brine.

| Salt | Mass (g) |
|---|---|
| CaCl₂ · 6H₂O | 16.6 |
| MgCl₂ · 6H₂O | 5.6 |
| KCl | 1.0 |
| NaCl | 112.5 |

A 1% v/v (10,000 ppm) stock of the surfactant was prepared in brine and used to create a concentration series of 0-250 ppm in each of the following (i) brine only (ii) 9:1 (v/v) brine:petroleum ether (b.p. 180-280° C.) (iii) 9:1 (v/v) brine:field oil. Samples were mixed well and equilibrated at 60° C., before being allowed to cool to ambient temperature.

A 0-250 ppm series of the surfactant in 6:4 (v/v) brine:petroleum ether (b.p 180-280° C.) and 6:4 (v/v) brine:field oil (b.p 180-280° C.) was then analysed. In this case, the samples were mixed well and equilibrated to 80° C., before being allowed to cool to ambient temperature.

For the samples containing only brine, the surfactant was added directly to the aqueous phase. The surfactant was added to the organic phase of the 9:1 matrix when hydrocarbon was present.

FIGS. 3-7 show graphical representations of the results for surfactant in brine, brine and petroleum ether, and brine and field oil at water cuts of 90% and 60%.

TABLE 4
Summary of the estimated CMCs of surfactant in
brine, brine and petroleum ether, and brine and field oil.

| Sample | Water Cut (%) | T (° C.) | CMC (ppm) |
|---|---|---|---|
| Brine | 100 | 60 | 70-80 |
| Brine: Petroleum | 90 | 60 | 50-60 |
| Ether | 60 | 80 | 30-40 |
| Brine: Field Oil | 90 | 60 | 70-100 |
| | 60 | 80 | 100-250 |

The presence of petroleum ether led to a lowering of the CMC compared to the brine-only samples. This effect was stronger the higher the proportion of petroleum ether was present, with the lowest CMC observed at the lowest (60%) water cut. This behaviour is typical of water soluble corrosion inhibitors, especially when the dosage is based upon the volume of total fluids. A higher concentration of the water-soluble corrosion inhibitor components will be present in the aqueous phase of the fluids in this case.

Conversely, the presence of field oil was shown to significantly increase the CMC. Again, this effect was more pronounced with a higher proportion of field oil present; the highest CMC was observed with a 60% water cut. This suggests that the surfactant partitions to the hydrocarbon and such losses in the aqueous phase mean a higher concentration of corrosion inhibitor is needed to achieve micelle formation.

### EXPERIMENT 3: THE EFFECT OF SOLIDS ON CORROSION INHIBITION

FIGS. 8 to 11 show the results of a corrosion inhibitor parasitisation test with a variety of solids. The CMCs of four commercial corrosion inhibitor formulations were determined in 1 M NaCl. 1.98 mL of the sample was transferred to a cuvette, to which was added 20 μL of the micelle detection marker. The cuvette was sealed and gently inverted to mix and read for optical signal. Data is presented as a Micelle Index (MI) of the sample: a normalized indicator of micelle presence, where a gained value above 0.5 signifies the occurrence of surfactant aggregation.

The CMC of all four inhibitor formulations was determined to be <100 ppm. Each chemical was then prepared as 100 ppm solutions in 1 M NaCl. A 20 mL sample of an inhibitor solution was transferred to a vial charged with a known mass of the test solid, sealed and gently agitated at hourly intervals for 5 hours. The solutions were then allowed to equilibrate for a further 15 hours before micelle analysis. Tested solids were selected based on a range of formation, corrosion and scale products likely to be observed in the field. The sand contained well packed silicon dioxide with low surface area. Experiments carried out using calcium and barium sulfate required the pre-saturation, and filtration to 0.45 μm, of the 1 M NaCl solution prior to use to negate the sparing solubility of the salts in water.

All chemicals showed varying losses of surfactant chemicals to the solids tested, except in the case of the tested sand. The sand, as well as having low surface area, is expected to have low metal ion content, particularly on its surface. Higher losses were observed in solids that contained metal ions.

The highest attritions were observed in Inhibitors 1-3, where formation clays and scale solids proved to be particularly effective at eliminating available chemical from the bulk. Corrosion by-products also presented alternative surfactant binding sites, with iron oxide affecting the test chemicals to a greater degree than iron sulphide. Inhibitor 1 performed best overall in the experiments but was susceptible to higher concentrations of formation clays.

The simple micelle detection method employed here showed there were variations in the extent to which different inhibitors responded to different types of solids and micelles and demonstrates that this method is informative in understanding the effects solids play in a field context.

1. A method of determining the suitability of corrosion inhibitors, or other additives in the presence of corrosion inhibitor, for a given fluid environment comprising: determining if there is a difference in the presence or level of micelles between a fluid sample to which corrosion inhibitor has been added either alone or to which corrosion inhibitor and at least one additional fluid additive or additives have been added.

2. A method according to claim 1 wherein the method of determining the presence of micelle comprises:
a) obtaining a fluid sample to which corrosion inhibitor is to be added;
b) adding to the fluid sample either corrosion inhibitor alone or corrosion inhibitor and at least one additional to the fluid additive or additives;
c) adding a marker solution comprising an optically detectable marker to the fluid sample;
d) determining the presence or level of micelle in the sample of the fluid; and
e) determining the suitability of corrosion inhibitor, or other additive, for a given fluid environment, based on the difference in the presence or level of micelles between fluid samples comprising corrosion inhibitor alone or corrosion inhibitor and at least one additional fluid additive or additives.

3. A method according to claim 2, wherein the fluid sample in mixed after step b).

4. A method according to claim 2, wherein the optically detectable marker is selected from a list comprising NanoOrange®, 9-diethylamino-5-benzo[α]phenoxazinone, 6-Dodecanoyl-2-Dimethylaminonaphthalene, N-(3-Trethylammonuimpropyl)-4-(6-(4-(Diethylamino) Phenyl Hexatrienyl) Pyridinium Dibromide and 2-Anilinonaphthalene-6-sulfonic acid, meropolymethines, pyridinium-N-phenolate betaines, phenoxazones, N,N-dialkylaminonaphthalenes, N,N-dialkylaminostyrenes, N,N-dialkylaminonitrobenzenes, coumarins, N,N-dialkylindoaniline, vinylquinoliums, and arylaminonaphthalene sulfonates.

5. A method according to claim 1, wherein presence of micelle is determined using laser diffraction, interferometry or imaging, spectroscopic means, hyperspectral imaging, or flow cytometry.

6. A method according to claim 2, wherein the fluid sample is taken from, or representative of, a fluid environment of a conducting and containment system used to screen test, produce, transport and process oil and/or gas and their products.

7. A method according to claim 1, wherein the fluid additive is an intended fluid additive comprising an alternative corrosion inhibitor, biocides, foamers, defoamers, paraffin control agents, emulsifiers, demulsifiers, anti-swelling agents, hydrate inhibitors, anti-caking agents, scale dissolvers or inhibitors, wetting agents, or wax control agents.

8. A method according to claim 1, wherein the fluid additive is an unintended fluid additive and comprises solid particles.

9. A method according to claim 8, wherein the solid particles comprise sand, kaolin, limestone, illite, iron (II)(Ill) oxide, iron(II) sulfide, barium sulfate, or calcium sulfate.

10. A method according to claim 1, wherein determining the presence of micelle in the sample of the fluid is performed after separation of the aqueous and hydrocarbon phases or wherein determining the presence of micelle in the sample of the fluid is performed without separation of the aqueous and hydrocarbon phases.

11. A method according to claim 10, wherein separation of the aqueous and hydrocarbon phases is performed by settling through density, centrifugation, heating, and/or chemical treatment.

12. A method according to claim 1, wherein a concentration series of corrosion inhibitor is created by adding an increasing concentration of corrosion inhibitor to two or more fluid samples, wherein the presence of micelle is determined for each of the two or more samples in the concentration series.

13. A method according to claim 1, wherein a concentration series of corrosion inhibitor is created by adding corrosion inhibitor sequentially to a fluid sample to create a concentration series, wherein the presence of micelle is determined after each sequential addition of corrosion inhibitor.

14. A method according to claim 12, wherein a concentration series of corrosion inhibitor alone and a concentration series of corrosion inhibitor and fluid additive at a fixed concentration is compared to determine the suitability of corrosion inhibitors, or fluid additive for a given corrosion inhibitor, for a given fluid environment, in a fluid conducting and containment system.

15. A method according to claim 1, wherein a concentration series of the second fluid additive is created by adding an increasing concentration of fluid additive to two or more fluid samples, wherein the presence of micelle is determined for each of the two or more samples in the concentration series.

16. A method according to claim 1, wherein a concentration series of fluid additive is created by adding corrosion inhibitor sequentially to a fluid sample to create a concentration series, wherein the presence of micelle is determined after each sequential addition of corrosion inhibitor.

17. A method according to claim 15, wherein corrosion inhibitor alone at a fixed concentration and a concentration series of second fluid additive and corrosion inhibitor at a fixed concentration is compared to determine the suitability of corrosion inhibitors for a given fluid environment in a fluid conducting and containment system.

18. A method according to claim 1, wherein the method further comprises the additional step of using the presence or level of micelles to determine corrosion inhibitor aqueous phase partitioning in a fluid sample and/or the presence or level of reverse micelle to determine corrosion inhibitor hydrocarbon phase partitioning in a fluid sample.

19. A method according to claim 1, wherein an aqueous fluid is added to the fluid sample to form a predetermined ratio of aqueous fluid to hydrocarbon fluid.

20. A method according to claim 19, wherein the aqueous fluid is water or a brine solution.

21. A method according to claim 8, wherein the predetermined ratio of aqueous fluid to hydrocarbon fluid is 0.5% to 99.5% of aqueous fluid.

22. A method according to claim 8, wherein the predetermined ratio of aqueous fluid to hydrocarbon fluid is 10%, 50%, and/or 90% of aqueous fluid.

23. A method according to claim 2, wherein the mixing of step e) comprises mixing and equilibrating to between 60° C. and 80° C., before being allowed to cool to ambient temperature.

24. A method according to claim 18, wherein a concentration series of corrosion inhibitor is created after addition of the aqueous fluid to form a predetermined ratio of aqueous fluid to hydrocarbon fluid.

25. A method according to claim 18, wherein the method comprises determining the presence of micelle formation in an aqueous phase and/or the presence of reverse micelle formation in a hydrocarbon phase.

26. A method of determining the suitability of corrosion inhibitors for a given fluid environment comprising:
a) obtaining a fluid sample to which corrosion inhibitor is to be added;
b) adding to the fluid sample either corrosion inhibitor alone or corrosion inhibitor and at least one additional to the fluid additive or additives;
c) adding a marker solution comprising an optically detectable marker;
d) determining the presence and level of micelle; and
e) determining the suitability of the combination of corrosion inhibitors and at least one additional fluid additive or additives for a given fluid environment based on the difference in the presence or level of micelles between a fluid sample comprising corrosion inhibitor alone and corrosion inhibitor and at least one additional to the fluid additive or additives.

27. A method of determining the suitability of corrosion inhibitors for a given fluid environment comprising:
a) obtaining a fluid sample to which corrosion inhibitor is to be added;
b) adding a corrosion inhibitor at, or to slightly above, its critical micelle concentration;
c) creating a concentration series of increasing amounts of at least one solid;
d) adding a marker solution comprising an optically detectable marker;
e) determining the presence of micelle; and
f) determining the suitability of corrosion inhibitors for a given fluid environment based on the difference in the presence of micelles between samples in the concentration series of increasing amounts of at least one solid in the presence of a corrosion inhibitor at, or close to, its critical micelle concentration and a control of corrosion inhibitor at, or close to, its critical micelle concentration alone.

28. A method of determining the suitability of corrosion inhibitors to at least partially partition to an aqueous phase for a given fluid environment comprising:
a) obtaining a fluid sample to which corrosion inhibitor is to be added;
b) creating a concentration series of increasing concentration of corrosion inhibitor;
c) adding a marker solution comprising an optically detectable marker;
d) determining the presence of micelle formation in the aqueous phase and/or the presence of reverse micelle formation in the hydrocarbon phase.

29. A method according to claim 28, wherein an aqueous fluid is added to the fluid sample prior to step b) to form a predetermined ratio of aqueous fluid to hydrocarbon fluid.

30. A method according to claim 29, wherein the aqueous fluid is water or a brine solution.

31. A method according to claim 28, wherein the fluid sample is mixed after addition of the aqueous fluid and equilibrated at a temperature of 50° C. to 90° C., before being allowed to cool to ambient temperature.
